(19) United States
(12) Patent Application Publication
(10) Pub. No.: US 2022/0040490 A1
(43) Pub. Date: Feb. 10, 2022

(54) Reagents and methods for cancer treatment using Magnetic particle

(71) Applicant: Tianxin Wang, Walnut Creek, CA (US)

(72) Inventor: Tianxin Wang, Walnut Creek, CA (US)

(73) Assignee: Tianxin Wang, Walnut Creek, CA (US)

(21) Appl. No.: 17/496,750

(22) Filed: Oct. 7, 2021

Related U.S. Application Data
(63) Continuation of application No. 15/952,236, filed on Apr. 13, 2018, now abandoned.
(60) Provisional application No. 62/485,387, filed on Apr. 14, 2017.

(51) Int. Cl. A61N 2/00; A61P 35/00; A61K 9/00; A61K 47/69; A61K 31/4745; A61K 9/51; A61K 41/00; A61K 9/08; A61K 9/19

(52) U.S. Cl. CPC A61N 2/002; A61P 35/00; A61K 9/0009; A61K 47/6929; A61K 31/4745; A61K 9/0019; A61K 9/5153; A61K 41/0052; A61K 9/08; A61K 9/19; A61K 9/5115

(57) ABSTRACT

Methods, system and reagents to treat cancer using magnetic particle are disclosed. The method comprises giving patient magnetic particle in combination with TLR agonist type composition and optionally later followed by immune check point inhibitor treatment at therapeutical effective amount. The magnetic particle and TLR agonist type composition is given by intratumoural injection.

### CROSS-REFERENCE TO RELATED APPLICATION

This application is a continuation application of U.S. application Ser. No. 15/952,236 filed on Apr. 13, 2018, which claims priority to U.S. Provisional Patent Application 62,485,387 filed on Apr. 14, 2017. The entire disclosure of the prior application is considered to be part of the disclosure of the instant application and is hereby incorporated by reference.

### FIELD

This disclosure provides magnetic particle, formulations comprising magnetic particle and Toll-like receptor (TLR) agonist, and methods of using the same for treating cancer.

### BACKGROUND

Despite recent advances in tumor therapy of solid tumors such as antibodies, the need for more efficacious and cost-effective treatment options remains. Thermotherapy or more specifically hyperthermia is an appealing approach for the treatment of cancer, as, compared to chemotherapy or radiation therapy, fewer side effects are expected for a wide range of tumor diseases due to its physical mode of action. However, currently available modalities are still suboptimal and warrant improvement.

One preferred modality of thermotherapy is a method wherein magnetic nanoparticles are directly introduced into a tumor. The nanoparticles are subsequently heated in an alternating magnetic field. Depending on the duration of treatment and the achieved intratumoral temperatures, the tumor cells are either directly destroyed (thermal ablation) or sensitized for concomitant chemo- or radiotherapy (hyperthermia). With this new procedure, it is possible to combat the tumor from inside out, thereby sparing surrounding healthy tissue. This treatment modality has shown promising therapeutic effects in the treatment of glioblastoma.

### SUMMARY

The current invention relates to novel methods and reagents to treat cancer using magnetic particle. The method comprises giving patient magnetic particle in combination with immune function enhancing agent such as TLR agonist type composition and optionally later followed by immune check point inhibitor treatment at therapeutical effective amount. The magnetic particle and immune function enhancing agent such as TLR agonist type composition is given by intratumoural injection. The immune function enhancing agent such as TLR agonist composition can be given to the patient by intratumoural injection as a mixture or sequentially (before or after) to the same tumor injected with patient magnetic particle. The method is based on the principle of introducing magnetic particles at micrometer or nanometer size directly into a tumor and then heating them in an alternating magnetic field to kill the cancer cell, generating in situ cancer vaccine to treat metastasis.

### DEFINITIONS

Unless defined otherwise, all technical and scientific terms used herein have the same meaning as commonly understood by one of ordinary skill in the art to which this disclosure belongs. As used herein the following terms have the following meanings.

It must be noted that as used herein and in the appended claims, the singular forms “a”, “an”, and “the” include plural referents unless the context clearly dictates otherwise. Thus, for example, reference to “an adjuvant” includes a plurality of adjuvants.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) claimed. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

As used herein, the term “treating” refers to preventing, curing, reversing, attenuating, alleviating, minimizing, inhibiting, suppressing and/or halting a disease or disorder, including one or more clinical symptoms thereof.

As used herein, the term “composition” refers to a preparation suitable for administration to an intended patient for therapeutic purposes that contains at least one pharmaceutically active ingredient, including any solid form thereof. In certain embodiments, the composition is formulated as an injectable formulation. In certain embodiments, the composition is formulated as a film, gel, patch, or liquid solution. As used herein, the term topically refers to administering a composition non-systemically to the surface of a tissue (e.g., a tumor) and/or organ (internal or, in some cases, external; through a catheter) to be treated, for local effect.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the desired tissue or a tissue adjacent to the desired tissue.

As used herein, the term “formulated” or “formulation” refers to the process in which different chemical substances, including one or more pharmaceutically active ingredients, are combined to produce a dosage form. In certain embodiments, two or more pharmaceutically active ingredients can be coformulated into a single dosage form or combined dosage unit or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “delivery” refers to approaches, formulations, technologies, and systems for transporting a pharmaceutical composition in the body as needed to safely achieve its desired therapeutic effect. In some embodiments, an effective amount of the composition is formulated for intratumoral injection into the patient (e.g., intratumoral delivery).

As used herein, the term “solution” refers to solutions, suspensions, emulsions, drops, ointments, liquid wash, sprays, liposomes which are well known in the art. In some embodiments, the liquid solution contains an aqueous pH buffering agent which resists changes in pH when small quantities of acid or base are added. In certain embodiments, the liquid solution contains a lubricity enhancing agent.

As used herein, the term “pH buffering agent” refers to an aqueous buffer solution which resists changes in pH when small quantities of acid or base are added to it. pH Buffering solutions typically comprise of a mixture of weak acid and its conjugate base, or vice versa. For example, pH buffering solutions may comprise phosphates such as sodium phosphate, sodium dihydrogen phosphate, sodium dihydrogen phosphate dihydrate, disodium hydrogen phosphate, disodium hydrogen phosphate dodecahydrate, potassium phosphate, potassium dihydrogen phosphate and dipotassium hydrogen phosphate; boric acid and borates such as, sodium borate and potassium borate; citric acid and citrates such as sodium citrate and disodium citrate; acetates such as sodium acetate and potassium acetate; carbonates such as sodium carbonate and sodium hydrogen carbonate, etc. pH Adjusting agents can include, for example, acids such as hydrochloric acid, lactic acid, citric acid, phosphoric acid and acetic acid, and alkaline bases such as sodium hydroxide, potassium hydroxide, sodium carbonate and sodium hydrogen carbonate, etc. In some embodiments, the pH buffering agent is a phosphate buffered saline (PBS) solution (i.e., containing sodium phosphate, sodium chloride and in some formulations, potassium chloride and potassium phosphate).

### DETAILED DESCRIPTION

The current invention relates to novel methods and reagents to treat cancer using magnetic particle. The method comprises giving patient magnetic particle in combination with immune function enhancing agent such as TLR agonist type composition and optionally later followed by immune check point inhibitor treatment at therapeutical effective amount. The magnetic particle and immune function enhancing agent such as TLR agonist type composition is given by intratumoural injection. The immune function enhancing agent such as TLR agonist composition can be given to the patient by intratumoural injection as a mixture or sequentially (before or after) to the same tumor injected with patient magnetic particle. The method is based on the principle of introducing magnetic particles at micrometer or nanometer size directly into a tumor and then heating them in an alternating magnetic field to kill the cancer cell, generating in situ cancer vaccine to treat metastasis.

The current invention also discloses novel formulations to treat cancer. The formulation comprises magnetic particle and immune function enhancing agent such as TLR agonist type composition in a pharmaceutical acceptable carrier. It can be injectable solution or solid dosage form such as lyophilized formulation that can be reconstituted into injectable solution. The formulation contains magnetic particle and immune function enhancing agent such as TLR agonist type composition as well as pharmaceutical acceptable excipients suitable for injection such as buffering salt (e.g. PBS salt), amino acid, carbohydrate (e.g. mannose, trehalose) and surfactant (e.g. PEG, tween, PVA, lethicin) or their combination.

The cancer cell killing is based on thermal ablation treatment of local tumors. The method is based on the principle of introducing magnetic micro or nanoparticles directly into a tumor and then heating them in an alternating magnetic field. For example, at approximately 15 nm˜10 um in diameter, the micro or nano particles, which are suspended in solution, the particles are activated by a magnetic field that changes its polarity up to 10,000˜500,000 times per second, generating heat. Examples of magnetic particle suitable for the current invention can be found in patent application EP20060742238, U.S. Pat. No. 8,688,229, PCT/EP2012/003381 (WO2013020701A3), U.S. Ser. No. 12/227,843 (US20090156976A1/U.S. Pat. No. 8,057,418B2), NanoTherm™ particles (from Magforce AG) and those in NanoTherm™ ferrofluid. The particles described in the prior art can be readily adopted for the current invention. The particles can switch their polarity ˜100,000 times every second in a magnetic field applicator (e.g. using the NanoActivator like device), which was developed specifically for this kind of therapy (e.g. NANOTHERM™ THERAPY, the machine's 100 kHz oscillating coil current can be continuously adjusted. The resulting magnetic field oscillates the iron oxide particles in the NanoTherm™ magnetic fluid, creating elevated therapeutic treatment temperatures within the tumor to kill cells).

The above cancer cell thermal ablation method uses paramagnetic particles activated by alternating magnetic fields. In the current invention the cancer cell thermal ablation method can also use colloidal metal, plasmonic, or conducting particles activated by electromagnetic radiation instead of using paramagnetic particles activated by alternating magnetic fields. In some embodiments, the particle may be a magnetic or paramagnetic (e.g., iron oxide particle) particularly when the energy source is an alternating magnetic field. In other embodiments, the particle may be a conducting material (e.g., gold or other metal colloids, nanoshells, nanorods, buckeyballs and carbon nanotubes), particularly when the energy source is radiowaves. In the examples and embodiments of the current invention the magnetic particles can be replaced with colloidal metal, plasmonic, or conducting particles activatable by electromagnetic radiation instead.

Examples of suitable immune check point inhibitors include antibody against PD-1, antibody against PD-L1, antibody against CTLA-4 or their combinations. Some are commercial available and can be readily used for the current invention such as Ipilimumab, Tremelimumab, Atezolizumab, Nivolumab and Pembrolizumab. They can be administered to the patient before or after the magnetic particle injection and magnetic field treatment. For example, the patient can be intravenously injected with Ipilimumab 3˜10 mg/kg every 3 weeks for 4 doses after treatment or Atezolizumab 1200 mg IV q3wk after treatment until disease progression. The current treatment dosing of these immune check point inhibitors can be used.

Examples of suitable immune function enhancing agent composition include pattern recognition receptor (PRR) ligands, TLR3 ligands, RLR ligands, TLR4 ligands, TLR5 ligands, TLR7/8 ligands, TLR9 ligands, Nod-Like receptor (NLR) ligands such as NOD2 ligands, C-Type Lectin Receptors (CLR) ligands or a combination thereof. The immune function enhancing agent can be a vaccine adjuvant. Preferably the Toll-like receptor ligand is a Toll-like receptors (TLR) agonist. Exemplary Toll-like receptors (TLR) agonists include, but are not limited to, CpG (CpG ODNs), poly IC, imiquimod, or a combination thereof. Many are commercially available (e.g. Invivogen). and can be readily used for the current invention. Example include imidazoquinoline family of TLR7/8 Ligands (e.g. imiquimod (R837), gardiquimod, resiquimod (R848), 3M-052, 3M-852, 3M-S-34240), CpG ODNs (CpG oligodeoxynucleotide) such as ODN 1826 and ODN 2216, synthetic analogs of dsRNA, such as poly IC (e.g. Poly ICLC, poly IC-Kanamycin, PolyI:PolyC12U), TLR4/5 Ligands such as Bacterial lipopolysaccharides (LPS, e.g. monophosphoryl lipid A), bacterial flagellin (e.g. Vibrio vulnificus flagellin B) or their derivatives, or their combinations.

They can be in form of active drug, prodrug, liposome, emulsion, gel, micelle, precipitate, suspension, conjugated to polymer drug carrier (e.g. dextran) or encapsulated in biodegradable micro particle/nano particle (e.g. those made of PLA, PLGA, PCL, PGA or PHB). The use and preparation of immune function enhancing agent encapsulated micro particle/nano particle or its prodrug are well known to the skilled in the art. Examples of them suitable for the current invention can be found in or adopted from US patent applications US 13/560,955 (US20130028941A1), U.S. Ser. No. 12/764,569 (US20110223201A1), U.S. Ser. No. 12/788,266 (US20110027217A1), publication in Vaccine. 2014 May 19;32(24):2882-95, Science. 2015 Jun. 19; 348(6241): aaa8205 and Nat Commun. 2016; 7: 13193. and their related citations.

Other molecules that can activate and/or boost the function of immune system and immune cells such as APC, B cells and T cells can also be incorporated into the formulation containing magnetic particles. Suitable immune function activating and/or boosting molecule can be selected from Granulocyte macrophage colony-stimulating factor (e.g. sargramostim or molgramostim), immunostimulatory monoclonal antibody (e.g. anti-MR antibody such as lirilumab, antibody for CD137 such as urelumab or utomilumab), FMS-like tyrosine kinase 3 ligand (FLT3L), other pattern recognition receptor agonists besides poly IC, CpG and imiquimod, T-cell-tropic chemokines such as CCL2, CCL1, CCL22 and CCL17, B-cell chemoattractant such as CXCL13, Interferon gamma, type I IFN (e.g. IFN-a, IFN-beta), tumor necrosis factor (TNF)-beta, TNF-alpha, IL-1, interleukin-2 (IL-2 such as aldesleukin, teceleukin or bioleukin), interleukin-10 (IL-10), IL-12, IL-6, IL-24, IL-2, IL-18, IL-4, IL-5, IL-6, IL-9 and IL-13 or their derivatives such as PEGylated derivative, CD1d ligand, Vα14/Vβ8.2 T cell receptor ligand, iNKT agonist, α-galactosylceramide (α-GalCer), α-glucosylceramide (α-GlcCer), α-glucuronylceramide, α-galacturonylceramide, Isoglobotriosylceramide (iGb3) and HS44. The agents can be added to the formulation described herein at a therapeutically effective amount, to be used as an intratumoral injection.

In one example, PLGA-R837 (R837 encapsulated in Poly Lactide-co-Glycolide particles) nanoparticle are prepared using o/w single-emulsion method. Briefly, R837 (TLR7 ligand) is dissolved in DMSO at 2.5 mg/ml. A total of 50 μL R837 is added to 1 ml PLGA (5 mg/ ml) dissolved in dichloromethane. Next the mixture is homogenized with 0.4 ml 5% w/v PVA solution for 10 min using ultrasonication. The o/w emulsion is added to 2.1 ml of a 5% w/v solution of PVA to evaporate the organic solvent for 4 h at room temperature. PLGA-R837 nanoparticles are obtained after centrifugation at 3,500 g for 20 min. Combination of immune function enhancing agent can also be encapsulated together in micro/nano particles. For example, R837 or R848 is dissolved in DMSO at 2.5 mg/ml. CpG ODN 2216 is dissolved in DMSO/H2O 1:1 at 50 mg/ml. 50 μl R837/R848 and 100 μl CpG ODN 2216 solutions are added to 1 ml mPEG-PLGA (10 mg/ml) dissolved in acetonitrile. Next, the mixture was dropwise added into 5 ml water containing 100 mg poly IC. After 1 h stirring and 12 h standing, the nanoparticles are obtained after centrifugation at 22,000 g for 5 min.

The said above biodegradable micro particle/nano particle can be the magnetic particle itself used to kill cancer cells with alternating magnetic field. Immune function enhancing agent type molecule can be directly conjugated to or absorbed to or encapsulated in the magnetic particles. When being encapsulated, preferably the magnetic particle is made of biodegradable materials. For example, the magnetic particles having surface amino group (e.g. those described in U.S. Ser. No. 07/968,158 (U.S. Pat. No. 5,466,609), PCT/EP2012/003381 and NanoTherm™ particles) can be mix with polylC or CpG OND or both in solution (e.g. 10:1˜1000:1 weight ratio). The polylC /CpG will bind to the surface of magnetic particles forming a complex, which can be used for the current invention either with or without removing the unbound polylC /CpG. The immune function enhancing agent type molecule can also be conjugated to the magnetic particles. For example, magnetic particles having surface —COOH group (e.g. introduced by carboxyl silane) can conjugate to the —OH of polylC, CpG and gardiquimod/resiquimod by forming ester bond.

Alternatively, a cleavable linker can be used to conjugate particle with immune function enhancing agent type molecule. For example, succinic acid can be used as linker to conjugate polyIC, CpG and gardiquimod with aminosilane coated magnetic particles. The synthesis of compound encapsulated magnetic particles is well known to the skilled in the art. These described in publications (e.g. those described in U.S. Ser. No. 07/968,158, PCT/EP2012/003381, PCT/GB1993/000952(WO1993023795A1)) can be readily adapted for the current invention to encapsulate the immune function enhancing agent type molecule inside the magnetic particles. Preferably the matrix of magnetic particle is biodegradable to release the encapsulated drug. There are many biodegradable polymers can be found in the publications to prepare micro or nano particles, which can be readily adopted for the current invention. In one example, PLGA coated magnetic particle are prepared using o/w single-emulsion method. Briefly, imiquimod is dissolved in DMSO at 5 mg/ml. A total of 100 μL R837 is added to 1 ml PLGA (10 mg/ ml) dissolved in dichloromethane. 1 mL 10mg/mL magnetic particle (10-50 nm in diameter) aqueous solution (e.g. those described in U.S. Ser. No. 07/968,158, PCT/EP2012/003381 or NanoTherm™ particles) is mixed with 3 mg polyIC and 3 mg class A CpG OND. Next the PLGA solution is homogenized with 0.5 ml resulting particle solution for 10 min using ultrasonication. The o/w emulsion is added to 2.1 ml of a 5% w/v solution of PVA to evaporate the organic solvent. The final magnetic particles are obtained after centrifugation at 3,500 g for 20 min.

Preferably the immune function enhancing agent type composition containing magnetic particles is given intratumorally at therapeutical effective amount. For example, the imiquimod can be given at the amount between 0.2 mg˜50 mg as free drug or given as 10 mg˜1000 mg micro or nano particle encapsulating 0.2 mg˜50 mg imiquimod. Other suitable dosing can be used, as long as it can produce satisfactory therapeutical effect, which can be determined experimentally by screening and testing with well-known protocol and methods.

As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective amount” are known to those of skill in the art and are described.

Dosage levels typically fall in the range of about 0.001 up to 100 mg/kg; with levels in the range of about 0.05 up to 10 mg/kg are generally applicable. A compound can be administered parenterally, such as intravascularly, intravenously, intraarterially, intramuscularly, subcutaneously, or the like. A dose can then be formulated in animal models to achieve the IC50 as determined in cell culture. Such information can be used to more accurately determine useful initial doses in humans. Levels of drug in plasma or tumor may be measured, for example, by HPLC. The exact formulation, route of administration and dosage can be chosen by the individual physician in view of the patient's condition. Compounds described herein can be administered as a pharmaceutical or medicament formulated with a pharmaceutically acceptable carrier. Accordingly, the compounds may be used in the manufacture of a medicament or pharmaceutical composition. Pharmaceutical compositions of the invention may be formulated as solutions or lyophilized powders for parenteral administration. Powders may be reconstituted by addition of a suitable diluent or other pharmaceutically acceptable carrier prior to use. Liquid formulations may be buffered, isotonic, aqueous solutions. Powders also may be sprayed in dry form. Examples of suitable diluents are saline solution (either isotonic or non-isotonic), standard 5% dextrose in water, or buffered sodium or ammonium acetate solution. Such formulations are especially suitable for parenteral administration. Compounds may be formulated to include other medically useful drugs or biological agents. The compounds also may be administered in conjunction with the administration of other drugs or biological agents useful for the disease or condition to which the invention compounds are directed.

The magnetic particle can further be coated with a cancer cell binding ligand to increase its targeting to cancer cell, which may allow intravenous (iv) injection instead of intratumoural injection. Small molecule ligand for cancer such as folic acid and RGD (arginylglycylaspartic acid) peptide/peptidomimetic can be used for cancer targeting (e.g. those described in Curr Med Chem. 2014;21(14):1618-30; Current pharmaceutical design 16(9):1040-54 and Journal of Amino Acids, Volume 2012 (2012), Article ID 967347). Folic acid or RGD peptide can be conjugated to the surface of the particle to increase cancer targeting. Administering the resulting particle to the patient can be used to treat cancer. Small protein ligand or antibody or antibody fragment for cancer can also be used to coat the magnetic particles. For example, Decorin, VEGF165b, VEGF antagonist in PCT/CA2010/000275 (WO2010102380A1) can be used to coat the magnetic particle's surface.

The current invention also discloses novel formulations to treat cancer. The formulation comprises magnetic particle and immune function enhancing agent type composition in a pharmaceutical acceptable carrier. It can be injectable solution or solid dosage form such as lyophilized formulation that can be reconstituted to injectable solution. The formulation contains magnetic particle and immune function enhancing agent type composition as well as pharmaceutical acceptable excipients suitable for injection. The immune function enhancing agent can be in form of active drug, prodrug, liposome, micelle, emulsion, gel formulation, implant, thermal phase changing formulation, insoluble, suspension, conjugated to polymer drug carrier (e.g. dextran) or coated on or encapsulated in biodegradable micro particle/nano particle. Suitable size of the particle is between 10 nm˜50 um.

The present disclosure provides compositions and formulations which typically comprise at least one pharmaceutically acceptable carrier. Pharmaceutically acceptable carriers are known to one having ordinary skill in the art may be used, including water or saline. As is known in the art, the components as well as their relative amounts are determined by the intended use and method of delivery. The compositions provided in accordance with the present disclosure are formulated as a solution for delivery into a patient in need thereof, and are, in particular, focused on intratumoral delivery.

Diluent or carriers employed in the compositions can be selected so that they do not diminish the desired effects of the composition. Examples of suitable compositions include aqueous solutions, for example, a saline solution, 5% glucose. Other well-known pharmaceutically acceptable liquid carriers such as alcohols, glycols, esters and amides, may be employed. In certain embodiments, the composition further comprises one or more excipients, such as, but not limited to ionic strength modifying agents, solubility enhancing agents, sugars such as mannitol or sorbitol, pH buffering agent, surfactants, stabilizing polymer, preservatives, and/or co-solvents.

In certain embodiments, a polymer matrix or polymeric material is employed as a pharmaceutically acceptable carrier. The polymeric material described herein may comprise natural or unnatural polymers, for example, such as sugars, peptides, protein, laminin, collagen, hyaluronic acid, ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic. In certain embodiments, compositions provided herein may be formulated as films, gels, foams, or and other dosage forms.

Suitable ionic strength modifying agents include, for example, glycerin, propylene glycol, mannitol, glucose, dextrose, sorbitol, sodium chloride, potassium chloride, and other electrolytes.

In certain embodiments, the solubility of the TLR agonist may need to be enhanced. In such cases, the solubility may be increased by the use of appropriate formulation techniques, such as the incorporation of solubility-enhancing compositions such as mannitol, ethanol, glycerin, polyethylene glycols, propylene glycol, poloxomers, and others known in the art.

Suitable pH buffering agents for use in the compositions herein include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or IVIES. In certain embodiments, an appropriate buffer system (e.g., sodium phosphate, sodium acetate, sodium citrate, sodium borate or boric acid) is added to the composition to prevent pH drift under storage conditions. In some embodiments, the buffer is a phosphate buffered saline (PBS) solution (i.e., containing sodium phosphate, sodium chloride and in some formulations, potassium chloride and potassium phosphate). The particular concentration will vary, depending on the agent employed. In certain embodiments, the pH buffer system (e.g., sodium phosphate, sodium acetate, sodium citrate, sodium borate or boric acid) is added to maintain a pH within the range of from about pH 4 to about pH 8, or about pH 5 to about pH 8, or about pH 6 to about pH 8, or about pH 7 to about pH 8. In some embodiments, the buffer is chosen to maintain a pH within the range of from about pH 2 to about pH 11. In some embodiments, the pH is from about pH 5 to about pH 8. In some embodiments, the buffer is a saline buffer. In certain embodiments, the pH is from about pH 4 and about pH 8, or from about pH 3 to about pH 8, or from about pH 4 to about pH 7.

Surfactants can be employed in the composition to deliver higher concentrations of cell surface anchoring antigen conjugates and immune function enhancing agents. The surfactants function to solubilize the insoluble and stabilize colloid dispersion, such as micellar solution, microemulsion, emulsion and suspension. Suitable surfactants comprise polysorbate, poloxamer, polyoxyl 40 stearate, polyoxyl castor oil, tyloxapol, triton, and sorbitan monolaurate. In one embodiment, the surfactants have hydrophile/lipophile/balance (HLB) in the range of 12.4 to 13.2 and are acceptable for ophthalmic use, such as TritonX114 and tyloxapol.

The compositions described herein may be sterilized to remove unwanted contaminants including, but not limited to, endotoxins and infectious agents. Sterilization techniques which do not adversely affect the structure and biotropic properties of the cell surface anchoring antigen conjugates can be used. In certain embodiments, the composition can be disinfected and/or sterilized using conventional sterilization techniques including propylene oxide or ethylene oxide treatment, sterile filtration, gas plasma sterilization, gamma radiation, electron beam, and/or sterilization with a peracid, such as peracetic acid. In one embodiment, the composition can be subjected to one or more sterilization processes. Alternatively, the composition may be wrapped in any type of container including a plastic wrap or a foil wrap, and may be further sterilized.

In some embodiments, preservatives are added to the composition to prevent microbial contamination during use. Suitable preservatives added to the anti-adhesion compositions comprise benzalkonium chloride, benzoic acid, alkyl parabens, alkyl benzoates, chlorobutanol, chlorocresol, cetyl alcohols, fatty alcohols such as hexadecyl alcohol, organometallic compounds of mercury such as acetate, phenylmercury nitrate or borate, diazolidinyl urea, diisopropyl adipate, dimethyl polysiloxane, salts of EDTA, vitamin E and its mixtures. In certain embodiments, the preservative is selected from benzalkonium chloride, chlorobutanol, benzododecinium bromide, methyl paraben, propyl paraben, phenylethyl alcohol, edentate disodium, sorbic acid, or polyquarternium.

Formulations contemplated by the present disclosure may also be for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the component in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In making pharmaceutical compositions that include magnetic particle and TLR agonist described herein, the active ingredient is usually diluted by an excipient or carrier and/or enclosed within such a carrier. When the excipient serves as a diluent, it can be a solid, semi-solid, or liquid material (as above), which acts as a vehicle, carrier or medium for the active ingredient. Thus, the compositions can be in the form of gels, powders, suspensions, emulsions, solutions, containing, for example, up to 30% by weight of the active compounds, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: wetting agents; emulsifying and suspending agents; and preserving agents such as methyl- and propylhydroxy-benzoates.

Gels are used herein refer to a semi solid, jelly-like material that can have properties ranging from soft and weak to hard and tough. As is well known in the art, a gel is a non-fluid colloidal network or polymer network that is expanded throughout its whole volume by a fluid. A hydrogel is a type of gel which comprises a network of polymer chains that are hydrophilic, sometimes found as a colloidal gel in which water is the dispersion medium. Hydrogels are highly absorbent and can contain a high degree of water, such as, for example greater than 90% water. In some embodiments, the gel described herein comprises a natural or synthetic polymeric network. In some embodiments, the gel comprises a hydrophilic polymer matrix. In other embodiments, the gel comprises a hydrophobic polymer matrix. In some embodiments, the gel possesses a degree of flexibility very similar to natural tissue. In certain embodiments, the gel is biocompatible and absorbable. In certain embodiments, the gel is administered to the patient prior to, during or after surgical intervention.

Liquid solution as used herein refers to solutions, suspensions, emulsions, drops, ointments, liquid wash, sprays, liposomes which are well known in the art. In some embodiments, the liquid solution contains an aqueous pH buffer agent which resists changes in pH when small quantities of acid or base are added.

Alternatively, exemplary formulations may comprise: a) magnetic particle and TLR agonist as described herein; b) pharmaceutically acceptable carrier; and c) hydrophilic polymer as matrix network, wherein said compositions are formulated as viscous liquids, i.e., viscosities from several hundred to several thousand cps, gels or ointments. In these embodiments, the cell surface anchoring antigen conjugates is dispersed or dissolved in an appropriate pharmaceutically acceptable carrier.

In certain embodiments, the magnetic particle and TLR agonist or a composition comprising the same, is lyophilized prior to, during, or after, formulation. In certain embodiments, the magnetic particle and TLR agonist, or a composition comprising the same, is lyophilized in a pharmaceutical formulation comprising a bulking agent, a lyoprotectant, or a mixture thereof. In certain embodiments, the lyoprotectant is sucrose. In certain embodiments, the bulking agent is mannitol. In certain embodiments, magnetic particle and TLR agonist, or a composition comprising the same, is lyophilized in a pharmaceutical formulation comprising mannitol and sucrose. Exemplary pharmaceutical formulations may comprise about 1-20% mannitol and about 1-20% sucrose. The pharmaceutical formulations may further comprise one or more buffers, including but not limited to, phosphate buffers. Accordingly, also provided herein is a lyophilized composition comprising a nanoparticle or composition comprising the same as described herein.

Suitable dosages can be determined by standard methods, for example by establishing dose-response curves in laboratory animal models or in clinical trials and can vary significantly depending on the patient condition, the disease state being treated, the route of administration and tissue distribution, and the possibility of co-usage of other therapeutic treatments. The effective amount to be administered to a patient is based on body surface area, patient weight or mass, and physician assessment of patient condition. In various exemplary embodiments, a dose ranges from about 0.01 mg to about 500 mg. In other illustrative aspects, effective doses ranges from about 0. 1 μg to about 1000 mg per dose, 1 μg to about 100 mg per dose, or from about 100 μg to about 50 mg per dose, or from about 500 μg to about 10 mg per dose or from about 1 mg to 10 mg per dose, or from about 1 to about 100 mg per dose, or from about 1 mg to 5000 mg per dose, or from about 1 mg to 3000 mg per dose, or from about 100 mg to 3000 mg per dose, or from about 1000 mg to 3000 mg per dose. In any of the various embodiments described herein, effective doses ranges from about 0.01 μg to about 1000 mg per dose, 1 μg to about 100 mg per dose, about 100 μg to about 1.0 mg, about 50 μg to about 600 μg, about 50 μg to about 700 μg, about 100 μg to about 200 μg, about 100 μg to about 600 μg, about 100 μg to about 500 μg, about 200 μg to about 600 μg, or from about 100 μg to about 50 mg per dose, or from about 500 μg to about 10 mg per dose or from about 1 mg to about 10 mg per dose. In other illustrative embodiments, effective doses can be about 1 μg, about 10 μg, about 25 μg, about 50 μg, about 75 μg, about 100 μg, about 125 μg, about 150 μg, about 200 μg, about 250 μg, about 275 μg, about 300 μg, about 350 μg, about 400 μg, about 450 μg, about 500 μg, about 550 μg, about 575 μg, about 600 μg, about 625 μg, about 650 μg, about 675 μg, about 700 μg, about 800 μg, about 900 μg, 1.0 mg, about 1.5 mg, about 2.0 mg, about 10 mg, about 100 mg, or about 100 mg to about 30 grams. In certain embodiments, the dose is from about 0.01 mL to about 10 mL.

In some embodiments, the formulations contain 20˜200 mg/mL magnetic micro or nano particles, 0.1˜50 mg/mL imidazoquinoline family TLR7/8 ligands (e.g. imiquimod or gardiquimod or resiquimod), 0.1˜50 mg/mL TLR3/RLR ligands (e.g. dsRNA such as poly IC or polyICLC), 0.1·50 mg/mL TLR9 ligands (e.g. CpG ODNs such as ODN 1826 or ODN 2216) in 1X PBS and then being lyophilized to give the final dosage form. In one example, the formulation contains 50 mg/mL magnetic micro or nano particles, 5 mg/mL imiquimod, 5 mg/mL poly IC and 5 mg/mL classe A CpG ODN 2216 in 1X PBS and 5% sucrose. In another example, the formulations contain 100 mg/mL magnetic micro or nano particles, 2 mg/mL imiquimod, 2 mg/mL poly IC, 2 mg/mL classe A CpG ODN 2216 and 0.1% tween-20 in 1X PBS and 5% mineral oil to form an emulsion. Surfactant (e.g. 0.1% tween-20) can be added to from stable suspension.

Yet in another example, the formulation is a solution containing 100˜200 mg/mL magnetic nano particles (e.g. the NanoTherm™ ferrofluid with an iron concentration of approximately 112 mg/mL) added with imiquimod (to 5 mg/mL final concentration), poly IC (to 2 mg/mL final concentration), CpG ODN 2216 (to 2 mg/mL final concentration). Suitable amount of surfactant can be added to stabilize the suspension. The formulation can be lyophilized to maintain its long term storage stability and be reconstituted before use. The dosage is 0.3 ml magnetic fluid injection per cm3 target tumor volume. The instillation of the nanoparticles is carried out either stereotactically, through transrectal ultrasound and X-ray guidance, interoperatively, or CT guided. The alternating magnetic current has a frequency of 100 kHz and produces a field strength between 2 and 15 kA/m. This magnetic current activates the iron oxide nanoparticles magnetic liquid and therapeutic treatment temperatures are achieved within the tumor.

Yet in another example, the formulation is a solution containing 100˜200 mg/mL magnetic nano particles (e.g. the NanoTherm™ ferrofluid with an iron concentration of approximately 112 mg/mL) added with 10 mg imiquimod per mL and 5 mg poly IC per mL. Suitable amount of surfactant can be added to from stable suspension.

Yet in another example, the formulation is a solution containing 100˜200 mg/mL magnetic nano particles (e.g. the NanoTherm™ ferrofluid with an iron concentration of approximately 112 mg/mL) added to each mL with 30 mg biodegradable (e.g. PLGA) micro or nano particles, which encapsulate 10 mg imiquimod and 5 mg poly IC. Suitable amount of surfactant can be added to from stable suspension.

Yet in another example, the formulation is a solution containing 100˜200 mg/mL magnetic nano particles (e.g. the NanoTherm™ ferrofluid with an iron concentration of approximately 112 mg/mL) added to each mL with 3 mg poly IC or 3 mg CpG ODN 2216 or both and 20 mg biodegradable PLGA nano particle encapsulating 5 mg imiquimod. Suitable amount of surfactant can be added to from stable suspension.

Yet in another example, the formulation is a solution containing 100˜200 mg/mL magnetic nano particles (iron oxide encapsulated in PLGA particle), which encapsulates 10% imiquimod by weight in the magnetic nano particles, is added to each mL with 3 mg poly IC or 3mg CpG ODN 2216 or both. Suitable amount of surfactant can be added to from stable suspension. After the patient receive the above the magnetic particle/magnetic field treatment, which uses an alternating magnetic current having a frequency of 100 kHz and a field strength between 2 and 15 kA/m, next the patient is intravenously injected with Ipilimumab 3˜10 mg/kg every 3 weeks for 4 doses, or Atezolizumab 1200 mg IV q3wk until disease stops progression.

Yet in another example, the formulation is a solution containing 100˜200 mg/mL magnetic nano particles, which (e.g. the NanoTherm™ ferrofluid with an iron concentration of approximately 112 mg/mL) is added to each mL with 3 mg poly IC or 3 mg CpG ODN 2216 or both and 20 mg biodegradable PLGA nano particles encapsulating 5 mg imiquimod. Suitable amount of surfactant can be added to from stable suspension. After the patient receive the above the magnetic particle/magnetic field treatment, the patient is intravenously injected with Ipilimumab 3˜10 mg/kg every 3 weeks for 4 doses, or Atezolizumab 1200 mg IV q3wk until disease stops progression.

Besides immune function enhancing agent, other molecules that can activate/boost the function of immune system and cell such as APC, B cell and T cells can also be incorporated in the intratumoral injection formulation. Suitable immune function activating/boosting molecule can be selected fromGranulocyte macrophage colony-stimulating factor (e.g. sargramostim or molgramostim), immunostimulatory monoclonal antibody (e.g. Anti-MR antibody such as Lirilumab, antibody for CD137 such as Urelumab or Utomilumab), FMS-like tyrosine kinase 3 ligand (FLT3L), other pattern recognition receptor agonists besides poly IC, CpG and imiquimod, T-cell-tropic chemokines such as CCL2, CCL1, CCL22 and CCL17, B-cell chemoattractant such as CXCL13, Interferon gamma, type I IFN (e.g. IFN-a, IFN-beta), tumor necrosis factor (TNF)-beta, TNF-alpha, IL-1, Interleukin-2 (IL-2 such as aldesleukin, teceleukin or bioleukin), interleukin-10 (IL-10), IL-12, IL-6, IL-24, IL-2, IL-18, IL-4, IL-5, IL-6, IL-9 and IL-13 or their derivatives such as PEGylated derivative. They can be added to the formulation described above at therapeutically effective amount, to be used as an intratumoral injection.

In another example, the formulation is a solution containing 100˜200 mg/mL magnetic nano particles, which (e.g. 20-50 nm size in diameter at 100 mg/mL concentration) is added to each mL with 3 mg poly IC or 3 mg CpG ODN 2216 or both, 20 mg biodegradable PLGA nano particles encapsulating 5 mg imiquimod, and granulocyte-monocyte colony-stimulating factor (10-200 μg). Suitable amount of surfactant can be added to from stable suspension. After the patient receive the above the magnetic particle with magnetic field treatment, the patient is intravenously injected with Ipilimumab 3˜10 mg/kg every 3 weeks for 4 doses, or Atezolizumab 1200 mg IV q3wk until disease stops progression.

In another example, the formulations is a solution containing 100˜200 mg/mL magnetic nano particles (iron oxide encapsulated in PLGA particle encapsulating 10% imiquimod, 20-100 nm size in diameter), 2 mg/mL poly IC, 2 mg/mL CpG ODN 2216, 50 μg/mL granulocyte-monocyte colony-stimulating factor, 1×10⁴˜1×10⁵ U/mL of IFN-α, 1-10 MIU/mL IL-2. After the patient receive the above the magnetic particle/magnetic field treatment, the patient is intravenously injected with Ipilimumab 3˜10 mg/kg every 3 weeks for 4 doses, or Atezolizumab 1200 mg IV q3wk until disease stops progression.

In another example, the formulation is a solution containing 100˜200 mg/mL magnetic nano particles, 2mg/mL poly IC, 2mg/mL CpG ODN 2216, 5 mg imiquimod, 25×10⁴ U/mL of IFN-α, 5 MIU/mL IL-2. After the patient receive the above the magnetic particle/magnetic field treatment, the patient is intravenously injected with Ipilimumab 3˜10 mg/kg every 3 weeks for 4 doses, or Atezolizumab 1200 mg IV q3wk until disease stops progression.

In another example, the formulation is a solution containing 100˜200 mg/mL magnetic nano particles, 2 mg/mL poly IC, 2 mg/mL CpG ODN 2216, 5 mg imiquimod, 5 MIU/mL IL-2. After the patient receive the above the magnetic particle/magnetic field treatment, the patient is intravenously injected with Ipilimumab 3˜10 mg/kg every 3 weeks for 4 doses, or Atezolizumab 1200 mg IV q3wk until disease stops progression.

1. A system to treat cancer comprising:
(a) a pharmaceutical composition comprising a magnetic particle and a Toll-like receptors (TLR) agonist, and
(b) a magnetic field applicator that can heat said magnetic particle with alternating magnetic field.

2. The system of claim 1, wherein the Toll-like receptors (TLR) is selected from TLR3, TLR5, TLR7, TLR8, TLR9 or their combination.

3. The system of claim 1, wherein the Toll-like receptors (TLR) agonist is CpG, poly IC, imiquimod, or a mixture thereof.

4. The system of claim 1, wherein the magnetic particle is coated with a cancer cell binding ligand selected from folic acid, RGD peptide, RGD peptidomimetic, TGF-α, GnRH, EGFR or VEGF antagonist.

5. The system of claim 1, wherein the Toll-like receptors (TLR) agonist is encapsulated in the magnetic particle.

6. The system of claim 1, wherein the Toll-like receptors (TLR) agonist is conjugated to the magnetic particle.

7. A method of treating tumor, comprising
(a) administering to a patient in need a therapeutically effective amount of pharmaceutical composition comprising a magnetic particle and a Toll-like receptors (TLR) agonist, and
(b) heating the magnetic particle with an alternating magnetic field.

8. The method of claim 7, wherein the treating and/or inhibiting comprises preventing metastasis of the tumor.

9. The method of claim 7, wherein the method comprises administering a therapeutically effective amount of an immune check point inhibitor.

10. The method of claims 7, wherein the administering is via intratumoral injection.

11. The method of claims 7, wherein the Toll-like receptors (TLR) agonist is CpG, poly IC, imiquimod, or a mixture thereof.
